(19) United States
(12) Patent Application Publication
(10) Pub. No.: US 2009/0306509 A1
(43) Pub. Date: Dec. 10, 2009

(54) FREE-HAND THREE-DIMENSIONAL ULTRASOUND DIAGNOSTIC IMAGING WITH POSITION AND ANGLE DETERMINATION SENSORS

(75) Inventors: Peder C. Pedersen, Sterling, MA (US); Thomas L. Szabo, Newburyport, MA (US)

Correspondence Address: BURNS & LEVINSON, LLP, 125 SUMMER STREET, BOSTON, MA, 02110, US

(73) Assignees: Worcester Polytechnic Institute, Worcester, MA (US); The Trustees of Boston University, Boston, MA (US)

(21) Appl. No.: 11/909,815

(22) PCT Filed: Mar. 30, 2006
(86) PCT No.: PCT/US2006/012327

§ 371 Date: Jun. 19, 2009

Related U.S. Application Data
(60) Provisional application No. 60/666,407, filed on Mar. 30, 2005.

(51) Int. Cl. A61B 8/14

(52) U.S. Cl. 600/446

(57) ABSTRACT

A freehand 3-D imaging system includes an integrated sensor configuration that provides position and orientation of each 2D imaging plane used for 3-D reconstruction without the need for external references. The position sensors communicate with the imaging system using either wired and wireless means. At least one translational and one angular sensor or three translational sensors acquire data utilized to compute position tags associated with 2D ultrasound image scan frames. The sensors can be built into the ultrasound transducer or can be reversibly connected and therefore retrofitted to existing imaging probes for freehand 3D imaging.

### STATEMENT REGARDING FEDERALLY SPONSORED RESEARCH OR DEVELOPMENT

This invention was made with Government support from the U.S. Army Medical Research Acquisition Activity under Contract No. DAMD17-03-2-0006. The Government has certain rights in the invention.

### BACKGROUND OF THE INVENTION

The present invention relates to ultrasonic imaging generally and more particularly to three-dimensional ultrasonic imaging using conventional two-dimensional ultrasonic imaging apparatus.

Over the last decade, 3D medical imaging has been playing an increasingly important role, in particular in computerized tomography (CT) and magnetic resonance imaging (MRI). The 3D reconstruction ability with these modalities has also improved over the same period of time. Given the method of CT and MRI scanning, the position of scan planes has been well defined. 3D ultrasound is now also finding widespread interest, where the most prominent specialty for 3D medical ultrasound imaging is in obstetrics, where the surface rendering methods have made very lifelike pictures of fetuses commonplace.

Examples of quantitative imaging applications utilizing 3D reconstruction are visualization of blood flow around tumors, planning and evaluating cancer treatment and cancer surgery, visualizing vessel structures (3D angiograms), seeing aneurisms and arterial plaques, reconstructive surgery, evaluation of cardiac function and guiding biopsy needles. These examples are independent of imaging modality used (CT, MRI, ultrasound), however, a position and angle registration system is required.

Five typical approaches to 3D medical ultrasound scanning are free hand scanning, mechanically vibrated linear array transducer, transducer with mounted sensor, two-dimensional transducer arrays, articulated scan arms, and cross-correlation of consecutive images.

A free hand scanning imaging system has no information about the true location and orientation of each scan plane relative to a reference location and orientation. However, the imaging system typically assumes that all the scan planes are parallel and equally spaced and furthermore, that the transducer is moved at constant and predetermined speed, so that the scan planes are at a known or presumed distance apart. This technique is widely used (such as Sonocubic for Terason), but it requires much operator training and cannot even in such cases be considered a quantitative imaging tool. Therefore, free-hand scanning is not a reliable technique for the above mentioned applications. The use of an articulated sensing arm for determining the position and orientation of the transducer at the end of an arm is not widely used now but was a primary way of constructing images in the early days of single element transducer ultrasound (see T. Szabo, “Diagnostic Ultrasound Imaging: Inside Out”, Elsevier Academic Press, Boston 2004.) The arm tracked the movement of the transducer, each position of the arm was used to determine the angle of ever acoustic line. The image was made up of the pulse-echo data from each line displayed in its proper angular orientation. Today, this method can be used to find the position and orientation of each 2D imaging plane.

Mechanically vibrated linear array transducer includes a linear array transducer that acquires individual scans of rectangular forms while it is being rotated over a specified angle. Thus, the scan volume is a sector in one cross-section and a rectangle in the orthogonal direction. Motor drives must be included within the transducer design, and consequently increase the size of the handle and cost of the probe and require motor driver power and software. This approach is a quantitative imaging technique, but with several limitations, such as not permitting Doppler imaging, not allowing 4D imaging (real time 3D ultrasound), and typically imaging only a small volume. Other variations include linear controlled or motorized translation of the probe and rotation of the probe circumferentially about a common axis.

Examples of commercially available triangulation position sensors for mounting on an ultrasound transducer for 3D ultrasound imaging registration are optical, electromagnetic or static discharge types. An electromagnetic version consists of a transmitter, placed on the transducer, and three receivers placed at different locations in the room (see Q. H. Huang, et al., “Development of a portable 3D ultrasound imaging system for musculoskeletal tissues”, Ultrasonics, 43:153-163, 2005.) From the phase shift difference in the received signals from these three receivers, the location and orientation of the ultrasound transducer can be determined. Such sensing methods require expensive equipment external to the sensing device for triangulation purposes; these can cause electromagnetic interference with other medical equipment commonly found in hospitals and clinics. An optical version is similar in nature to the electromagnetic system except that optical sensors and sources with higher precision are used. The optical system does not have the drawback of electromagnetic interference (see G. M. Treece, et al., “High definition freehand 3D ultrasound”, Ultrasound in Medicine and Biology, 29(4):529-546, April 2003.) From the phase shift difference in the received signals from these three receivers, the location of the ultrasound transducer can be determined. Such sensing methods require expensive equipment external to the sensing device for triangulation purposes; these can cause electromagnetic interference with other medical equipment commonly found in hospitals and clinics. An optical version is similar in nature to the electromagnetic system except that optical sensors and sources with higher precision are used. A further disadvantage of these sensor types is the fact that the scanning room must have these sensors installed and the system calibrated, before actual scanning can occur.

An alternative registration device is motor-driven mechanical scanning of the ultrasound transducer. All methods provide sensing or control of the positions of the transducer during the acquisitions of image planes. These methods involve a physical constraint that limits movement of the transducer to a prescribed direction or rotation.

Two-dimensional array transducers typically contain an M×N rectangular arrangement of array elements, in contrast to the conventional linear array which is a 1×N array. However, sparse two-dimensional transducer arrays have reduced resolution due to the reduced number of array elements. Fully populated 2D arrays, now commercially available, have good resolution but a small field-of-view compared to freehand imaging, where the field-of-view is determined by the length of the scan path. Also, cost of two-dimensional array transducers is another limiting factor along with the small volume that can be imaged (same limitation as the mechanically vibrated transducer).

Cross-correlation of consecutive images is a software method, which may be used in connection with freehand technique. It associates the degree of decorrelation in 2D cross-correlation of consecutive scans with the amount of displacement. The method is computationally demanding, cannot work with non-parallel scan planes, and cannot differentiate movement to the left from movement to the right.

Generally, three dimensional ultrasound (3D ultrasound) consists of combining information from a sequence of closely spaced scan planes; these scan planes are typically parallel, but they can also be oriented in a radial fashion when a mechanically scanned transducer is used. In freehand scanning, depending on the skills of the operator, the scan planes may deviate from parallel to a greater or smaller extent, the spacing between planes may depend on the uneven rate of handheld translation and the alignment of the planes may depend on the straightness of the manual scanning. The 3D reconstruction software typically carries out surface rendering, which means that surfaces with easily discernible features are created from contours in individual planes.

Alternatively, the 3D reconstruction software can produce what is referred to as “volume rendering” in which surfaces are displayed as semi-transparent to allow visualization of interior objects. 3D ultrasound is implemented in two forms: free-hand 3D ultrasound scanning and 3D ultrasound scanning with registration. Accurate surface rendering and volume rendering are very difficult to achieve with free-hand scanning even by skilled operators.

With free-hand 3D ultrasound scanning, the operator of the scanner moves the transducer, in a presumed straight path and with a presumed constant angle to the skin surface with as constant and specified velocity over the surface as possible. However, the software typically assumes the scan planes to be equally spaced with a known or presumed spacing. As this scanning requirement seldom is met, the result of the reconstruction is distorted.

In 3D ultrasound scanning with registration, the exact location of each scan plane is determined by a positioning device that typically is unrelated to the ultrasound scanner. For 3D ultrasound scanning with registration, the reconstruction software obtains a 3D position tag with each scan planes, which allows an accurate, or quantitative, reconstruction.

However, many applications require an accurate surface rendering to be carried out. Examples include a quantitative assessment of the size of cardiac defects, the extent of a cancerous lesion, the size of a deep vein thrombosis, the extent of an atherosclerotic plaque, the contours of a blood filled region due to trauma, the size of a flaw in a pressure vessel. High quality results for these applications cannot be easily achieved with free hand 3D ultrasound with known techniques. 3D ultrasound with registration provides better results, however significant work is still needed in the development of image processing algorithms.

An equally significant benefit of 3D ultrasound with registration is the ability to do accurate volumetric evaluations (quantitative volume rendering). Without registration, the length, straightness and direction of the manual scan path are unknown; therefore volumes cannot be estimated accurately.

### SUMMARY OF THE INVENTION

The present invention seeks to provide a free-hand, registration system for ultrasonic imaging, which is characterized by simplicity of construction and operation and relatively low cost. The system may be implemented in original equipment or as a retrofit to existing equipment having only two-dimensional (2D) imaging capabilities. Position tags (the term “position tag” is used inclusively herein to include position data and, where appropriate, orientation/angle data) associated with 2D image planes are computed from a variety of sensor configurations, all of which may be output to ultrasound image display programs for volumetric rendering by known interpolation techniques which typically form a sequence of ultrasound image planes with equal spacing and fixed lateral positioning or other suitable geometries for interpolation. The invention, thus, permits improved ultrasound scanning accuracy by reducing or eliminating variations in the scanning process introduced by a number of factors, including non-uniform scanning by a user, as well as sensor-dependent errors due to manufacturing variation, drift and hysteresis.

In a first aspect, the invention provides free-hand, ultrasonic imaging registration system having a transducer probe including a probe housing and a conventional ultrasound (for example, linear) array transducer operatively disposed in the probe housing that supplies ultrasound waves to a region of interest such as, for example, the abdominal region of a pregnant woman. The ultrasound transducer receives over time ultrasound waves reflecting from the region of interest as a plurality of transducer signals that can be converted into two dimensional (2D) image planes, wherein each of the received transducer signals has an associated image acquisition time.

In a first embodiment of the invention, one or more position sensors and one or more angle sensors are operatively integrated within or outside of the probe housing. As the term is used herein, “integrated” is intended to mean alternative options of formation as a unitary structure with the probe housing or, as noted above, reversibly connected to the housing so as to permit retrofitting of a conventional transducer probe with the position and angle sensors. The one or more position sensors acquire, as a function of time, position data for the probe, in one, two or three translational degrees of freedom, relative to an initial reference position, converting the acquired data into position signals. Similarly, the one or more angle sensors acquire, as a function of time, orientation data for the probe in one, two or three rotational degrees of freedom relative to a reference orientation and a starting time, converting the acquired angular data into at least one angular signal. The position and angular signals are communicated from the sensors to a “registration” processor, preferably through standardized data communications connections (e.g., USB, RS-232) and protocols (e.g., TCP/IP.) The signals may additionally or alternatively be communicated via wireless communication circuitry and protocols. The processing unit receives the position and angle signals, and associated ultrasound image acquisition timing data, and computes from the received information a position tag for each of the 2D ultrasound image planes acquired by the transducer array.

In a second embodiment, the present invention provides a free-hand, 3D ultrasound imaging registration system including transducer probe having a probe housing and a conventional ultrasound (for example, linear) array transducer, and one or more position sensors operatively integrated within or outside of the probe housing and acquiring, as a function of time, position data for the probe in three translational degrees of freedom, relative to an initial reference position and starting time. Similarly, the acquired position data is converted into at least one position signal and communicated from the one or more sensors to a registration processor, which in turn receives the position signal(s), as well as the transducer signals and associated ultrasound image acquisition timing data, and computes from the received information a position tag for each of the 2D ultrasound image planes acquired by the transducer array.

The ultrasound imaging registration systems and methods described are unique relative to registration methods presently available, in that the position and angle sensors acquire their respective data without the assistance of external position or orientation references (i.e., the data sensing is internal to the transducer probe, eliminating the need of some existing systems to perform triangulation with external sources.)

In another embodiment, one or more position sensors acquire the position data in three translational degrees of freedom, and one or more angle sensors acquire the angular data in three rotational degrees of freedom. This provides the registration processing unit with sufficient data (even redundant in some cases) to compute a 3D position tag. A three-axis microelectromechanical accelerometer with additional integration, for example, may be utilized as the position sensor, and a three-axis gyroscope may be employed as the angle sensor with additional integration, in order to acquire data in a complete six degrees of freedom.

In another aspect, the present invention provides a method of transducer probe registration for 3D ultrasound scanning including the step of providing a sensor-equipped ultrasound transducer probe according to the first embodiment described above, and acquiring as a function of time position and angular data via the position and angular sensors. Transducer array data are also acquired as a function of time, from which a sequence of 2D ultrasound image planes are normally derived by the imaging system. The position and angle position tag data are converted into signals that are transmitted to the imaging system via hard wired or wireless communications circuits and protocols. The registration processing unit computes the position tags by extracting the position data and angular data from the position signal(s) and angular signal(s), respectively, and deriving synchronous position tag coordinates from geometric transformations of the position data and orientation data relative to the reference position and orientation as a function of time with reference to a clock. The processor then associates each 2D image plane with position tag coordinates by comparing the image acquisition time associated with each 2D image plane with timing data corresponding to said position tag coordinates. Several techniques may be utilized to acquire timing information, including generating timing data internally to the transducer probe, or through synchronized sampling of asynchronously transmitting sensor and transducer array data. Alternatively, position data can be supplied on request by the imaging system coincident with each 2D imaging frame.

In yet another aspect, the present invention provides a method of transducer probe registration for 3D ultrasound scanning including the step of providing a sensor-equipped ultrasound transducer probe according to the second embodiment described above, and acquiring as a function of time position data via the position sensors along three translational degrees of freedom. Transducer array data are also acquired as a function of time, from which a sequence of 2D ultrasound image planes are derived by the imaging system. The acquired position tag data is converted into signals that are transmitted to the imaging system via hard wired or wireless communications circuits and protocols. The registration processing unit computes the position tags by extracting the position data from the position signal(s), and deriving synchronous position tag coordinates from geometric transformations of the position data relative to the reference position as a function of time with reference to a clock. The processor then associates each 2D image plane with position tag coordinates by comparing the image acquisition time associated with each 2D image plane with timing data corresponding to said position tag coordinates. Several techniques may be utilized to acquire timing information, including generating timing data internally to the transducer probe, or through synchronized sampling of asynchronously transmitting sensor and transducer array data. Alternatively, position data can be supplied on request by the imaging system coincident with each 2D imaging frame.

The position sensor(s) are of a type that acquires data along a single or multiple axes, including, but not limited to, optical sensors, self-contained electromagnetic sensors, and capacitive MEMS devices. In a preferred embodiment the position sensor comprises one or more light source(s) for illuminating the region of interest with sufficient intensity such that light reflects from the region of interest, an optical imaging means including at least one lens disposed in or upon the probe, so as to receive light reflected from the region of interest in the form of an optical image, and a light-sensitive image capture device for converting the optical image output from the lens into said position signal such as, for example a charge coupled device camera and digital signal processor. The light may be coupled to the image capture device through an appropriately designed optical fiber bundle. Several alternative designs of such an optical sensor will be described below. By optically acquiring images of the surface of a region of interest, and thus information regarding the position of the transducer probe relative to the region of interest or, alternatively stated, to reference position, the acquisition of positional information is much less sensitive to noise occurring during movement of the transducer probe. The optical path between the scanned skin surface and the unit in the transducer probe is relatively short and is not easily disturbed. This enhances the accuracy of the detected position of the transducer probe and thus also the quality of the three-dimensional ultrasound image resulting from a composition of two-dimensional slices based on said positional information.

The angle sensor(s) are of a type that senses rotation about a single or multiple axes, including, but not limited to, capacitive MEMS devices, gyroscopes, sensors employing the Coriolis force, and accelerometers.

In yet another embodiment, the present invention additionally provides a sensor calibrator that corrects for misalignment between the coordinate frame of the sensors and that of the imaging plane. Upon initial determination of the misalignment, a geometric factor can be utilized to correct for sensor to image plane misalignment.

In another embodiment, the present invention additionally provides means and method for compensating for sensor errors due to changes in the state of a sensor such as, for example, errors resulting from temperature drift and/or hysteresis.

### BRIEF DESCRIPTION OF THE FIGURES

For a better understanding of the present invention, together with other and further objects thereof, reference is made to the accompanying drawing and detailed description, wherein:

FIG. 1 is a block diagram of functional components one embodiment of an ultrasound imaging registration system in accordance with the present invention;

FIG. 2 is a schematic illustration of an embodiment of the present invention utilizing an optical position sensor; and

FIG. 3 is a functional block diagram illustrating a method of use of the present invention.

### DETAILED DESCRIPTION OF PREFERRED EMBODIMENTS OF THE INVENTION

### Overview

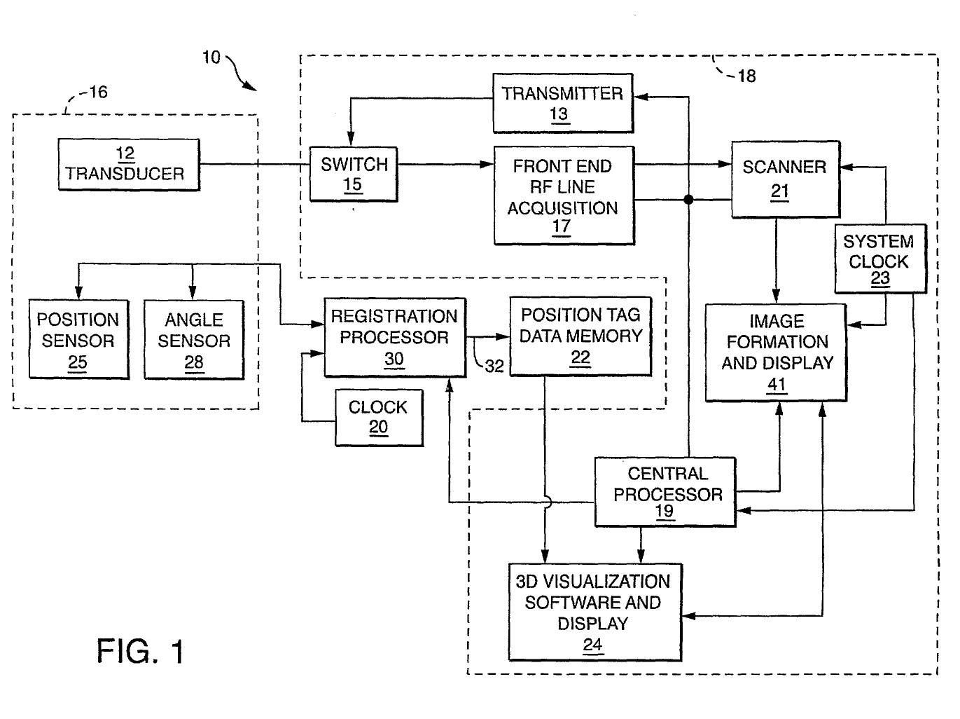

FIG. 1 shows a free-hand ultrasound medical diagnostic imaging system 10 within which is a first embodiment of an ultrasound registration system. An ultrasound imaging system sends excitation signals through a transmitter 13 through a switch 15 to the transducer 12 operatively disposed in a probe housing 16. The ultrasound array transducer 12 detects response echoes from a region of interest within a patient's anatomy. The imaging system receives echoes from the transducer 12 through the switch 15 that routes the signals to a front end 17 from where they are sent by a central processor 19 in synchronization with a system clock 23 to a scanner 21. From the scanner 21, processed signals are sent to the image formation and display section 41 from which 2D image frames are formed in synchronism with the system clock 23. The registration system includes, preferably, a system clock 20, memory 22 for storing position tags (described below) associated with each 2D ultrasound image plane acquired by transducer 12 and front end acquisition section 17 of the imaging system. Imaging system 10 further includes 3D visualization software and display system 24. The registration system includes various configurations of angle and position sensing elements operatively integrated within or upon probe housing 16. As the term is used herein, “integrated” is intended to mean that the angle and/or position sensing elements may be formed as a unitary structure with probe housing 16, or may be reversibly connectable to the probe housing such as, for example, through use of straps, clips or other fixation means. In the configuration depicted, the probe housing is equipped with one or more position sensors, such as position sensor 25, and one or more angle sensors, such as angle sensor 28. The registration system includes means 32 for communicating, respectively, the transducer signals from transducer 12 and position and angle signals from position sensor 25 and angle sensor 28 from the probe housing 16 to the front end section 17 and a registration processing unit or processor 30. The phrase “registration processing unite” is used herein interchangeably with the term “processor”, however, it will be understood by those of skill in the art that the invention is not limited to specific hardware configuration. In fact, the position and angular signal processing described herein could be performed by software executing on a processor integral to the transducer probe, a processor physically separated from the transducer probe and from the 3D visualization system 24, or on a processor integral to the 3D visualization system. In fact, signal processing functionality could be directly implemented completely in hardware at any of these physical locations.

In a method according to present invention, registration processor 30 is adapted to receive timing information associated with the 2D planes from the central processor 19 of the imaging system and the position signals and angular signals, from which processor 30 computes a position tag for each of the 2D image frames. It is worth noting that the sensors utilized in the present invention require no external references to generate the position and angular signals. The imaging system includes the central processor 19, system clock 23, switch 15, transmitter 13, front end rf line acquisition section 17, scanner 21, image formation and display section 41, position tag data memory 22 and 3D visualization software and display 24. The imaging system 18 is connected to the transducer 12 and registration processor 30. The registration system includes the registration processor 30, clock 20, and position sensor(s) 25 and angle sensor(s) 28.

As noted above, the illustration in FIG. 1 of registration processing unit 30 as a functional block distinct from the 3D visualization system 24 is representative of only one configuration. In certain alternative embodiments, the registration processor 30 is mounted within a compartment, or upon an exterior surface, of probe housing 16. In such embodiments, communications means 32 instead transmits the position tags to the memory 22. Communication means 32 may be comprised of wired connections using standard data communications interface protocols and physical connections (USB, serial), and/or may be comprised of wireless communications circuitry and protocols. In alternative configurations, registration processing unit 30 may actually be a processor of the 3D visualization system 24 or of the image acquisition system 18.

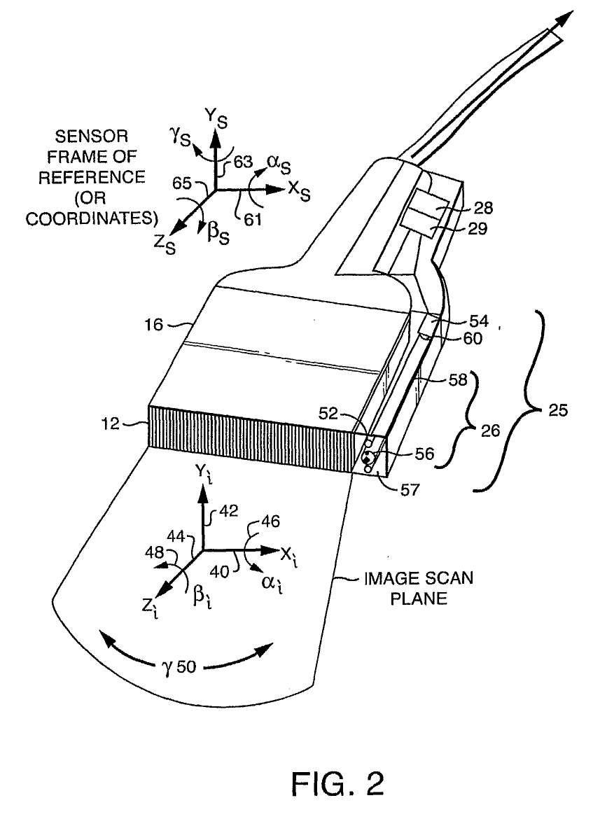

In another embodiment of the registration system, the at least one position sensor 25 operates so as to acquire position data along all three translational degrees of freedom (shown in FIG. 2 as orthogonal axes 40,42,44), but the angular sensors are optional.

Sensing Elements

Multiple position sensors may be utilized, any or all of which may comprise single-axis or multiple-axes sensors acquiring probe position data in one or more translational degrees of freedom. Similarly multiple angle sensors may be utilized, any or all of which may be capable of sensing rotation about a single or multiple axes. The position sensors may be optical sensors, self-contained electromagnetic sensors, capacitive MEMS devices and the like. Exemplary angle sensors include MEMS devices, gyroscopes, accelerometers, sensors that sense the Coriolis force, and the like. In certain embodiments, redundant data is obtained by utilizing multiple sensors acquiring data in overlapping translational or rotational degrees of freedom. Such redundant data may be utilized to achieve more accurate measurements and resultant 3D reconstructions. Depending upon the type of position sensor utilized and the amount of processing available in the sensor module, however, some data manipulation of the sensor output data may be necessary prior to its use by processor 30. With reference to FIG. 2, if for example, the position sensor employed is a microelectromechanical systems (MEMS) accelerometer 29, additional signal/data processing will be required to convert, through double integration, the sensed acceleration output of the accelerometer 29 into position data. This may be accomplished by the registration processor 30. An implementation of double integration signal processing is described by Lee, Seungbae, et al., “Two-Dimensional Position Detection System with MEMS Accelerometer for MOUSE Applications”, IEEE Transactions on Very Large Scale Integration (VLSI) Systems, Vol. 13, Issue 10, October 2005, the contents of which are hereby incorporated by reference in their entirety.

Position sensors 25 and 28 (illustrated as optical imaging means and an accelerometer) operate so as to acquire, as a function of time, position data of the ultrasound probe 16 in at least one of the three translation degrees of freedom 40,42,44 shown, relative to an initial reference position and starting time. Optical position sensor 25 is comprised of at least one light source 52 (e.g., a direct LED or laser diode couple to an optical fiber) for illuminating the region of interest with light of sufficient intensity that light reflects from the region of interest, an optical imaging means 26 including at least one lens 56 disposed in or upon the probe housing 16 (shown disposed in a compartment 57) so as to receive light reflected from the region of interest in the form of an optical image, and a light-sensitive image capture device 54 for converting the optical image output from lens 56 into a position signal. Capture device 54, in a preferred embodiment, is further comprised of a CCD camera at a relatively high capture rate relative to the sonographer's movement of the transducer and a digital signal processor (DSP) chip for converting the raw sensor images into one or more position signals indicating the transducer's motion in two translational degrees of freedom. The output of lens 56 is optically coupled to an optical fiber 58, and another lens 60, providing an optical path for and focusing of the reflected image onto the capture device 54.

During operation, the light source (or sources) 52 is preferably positioned at an angle α relative to lens 56 of optical imaging means 26. The angle can be any angle between 0° and 90°, but by illuminating the region of interest under a small angle the surface (i.e., skin) roughness in the optical image is enhanced. Preferably, the angle is between 20° and 60°, but the present invention is not to be limited to any range of angles.

Cross-correlation technology has been developed, related to optical mouse movement tracking, for optically detecting motion by directly imaging as an array of pixels the various particular spatial features of a surface below an optical source, such as an infrared (IR) light emitting diode (LED) and an image capture device. See Gordon, et al., U.S. Pat. No. 6,433,780, and Ross, et al., U.S. Pat. Nos. 5,578,813, 5,644,139 and 5,786,804, the contents of each of which are hereby incorporated herein by reference. Utilization of similar techniques results in the generation of the position signals that are transmitted from sensor 25 to registration processor 30. In an implementation of the invention reduced to practice by the applicants, and described below, an optical sensor with a DSP-processor was used, in the form of Agilent Technology Inc.'s ADNS-2610. This sensor is found in many optical computer mice, and is comprised essentially of a CCD camera that acquires images of a surface at a very high rate (1500 fps) and a DSP algorithm that makes a cross-correlation between consecutive images. By using the cross-correlation algorithm, the distance the optical sensor has moved was determined.

Angle sensor 28 (illustrated as a micro gyroscope) operates so as to acquire, as a function of time, angular data of the ultrasound probe in at least one of the three rotational degrees of freedom 61,63,65 shown, relative to an initial reference orientation and a starting time. Angle sensor 28 converts the acquired angular data into one or more angular signals that are transmitted to the registration processor 30.

2D and 3D Ultrasound Scanning with Registration

With reference again to FIG. 1, in operation, the imaging system transmitter 13 generates electrical signals for output to the transducer 12. The transducer 12 converts the electrical signals into an ultrasound transmit wave-pattern. Typically, the transducer 12 is positioned in contact with the skin and adjacent to a patient's anatomy. The transmit wave-pattern propagates into the patients anatomy where it is refracted, absorbed, dispersed and reflected. Reflected components propagate back to the transducer 12, where they are sensed by the transducer 12 and converted back into one or more electrical transducer signals and transmitted back to the imaging system front end 17. The degree of refraction, absorption, dispersion and reflection depends on the uniformity, density and structure of the encountered anatomy. The 3D reconstruction/visualization system 24 can register the exact location of limited field of view, so that closely spaced ultrasound 2D image scan planes with the position tags output by the registration system of the present invention can be used to define an enlarged 2D or a 3D image. First, echo data is received and beamformed to derive one or more limited field of view frames of image data while a sonographer moves the transducer along a patients skin surface. Second, registration of the 2D image planes may occur using the position tags, each 2D image plane having associated with it a position tag. A resulting image may then be obtained using conventional 3D interpolation and visualization techniques and/or by projecting the 3D volume onto a 2D plane.

For further discussion of the principles and techniques of 2D and 3D ultrasound, generally, see co-inventor Thomas L. Szabo's “Diagnostic Ultrasound Imaging: Inside Out”, Elsevier Academic Press, Boston 2004, the contents of which are hereby incorporated by reference in their entirety, and for a more detailed treatment of 3D image reconstruction from 2D scan planes or frames, see Q. H. Huang, et al., “Development of a portable 3D Ultrasound Imaging System for Musculoskeletal Tissues”, Ultrasonics, 43 (2005) 153-163, also incorporated by reference.

The sensors described permit continuous tracking of the transducer probe in multiple degrees of freedom during free-hand scanning. In a preferred embodiment, the one or more position sensors acquire the position data in all three translational degrees of freedom 40,42,44 (as could be accomplished with a three-axis MEMS linear accelerometer with integration to sense the depth axis), and the one or more angle sensors acquire the angular data in all three rotational degrees of freedom 61,63,65 (as could be achieved with a rotational three-axis gyroscope.) This permits the registration processor 30 to compute a 3D position tag for each of the 2D ultrasound image planes or frames.

Several imaging system operating modes may be implemented, characterized by the manner in which the position tags as a function of time are output to the storage memory 22 and visualization and display system 24. In a first mode, each of the sensors utilized (e.g., position sensors 26,29 and optionally angle sensor 28) is asynchronously transmitting its output in real-time to the registration processor 30, as is the imaging system 18, which sends timing signals associated with the creation of each 2D imaging frame to the registration processor 30. Registration processor 30 samples at regular sampling intervals each of these data streams to associate a particular data acquisition time with the acquired signals and image frames. Alternatively, in a second mode, registration processor 30 actively responds with position tag data to requests from the imaging system. The interrogation request may be synchronous with the completion of an ultrasound transducer array scan of the region of interest. Timing for each of these activities is supplied to registration processor 30 by reference clock 20 that, as noted above, may also be integrally disposed within or upon the transducer probe housing, or may be disposed off the probe.

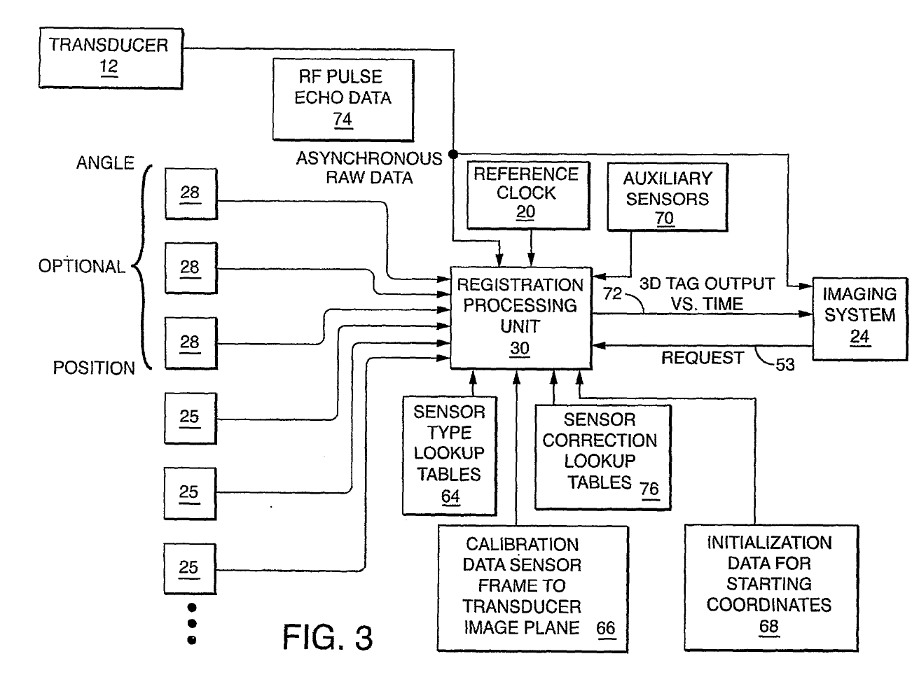

The function of registration processor 30 in computing position tags and in performing additional, optional tasks will now be described with reference to FIG. 3. Processor 30 receives the position signals from one or more position sensors 25 and angular signals from one or more angle sensors 28 (in embodiments equipped with angle sensors.) Processor 30 then identifies the type of sensor (e.g., translational or rotational, accelerometer or displacement) from a lookup table 64, and obtains the position and/or orientation data and performs the appropriate geometric transformation according to the received signals' sensor type, placement and orientation (i.e., in association with the physical coordinate axis or axes with which the sensor is aligned) to acquire the position tag. If, for example, the sensor is an accelerometer, a magnitude of the acceleration and a double integration with respect to time are computed to obtain displacement or position data (as cited above, a method is described in Lee et al., 1998.)

Registration processor 30 preferably also compensates the obtained position data for sensor misalignment (e.g., due to manufacturing variability) by a fixed geometric coordinate transformation according to calibration data (in a sensor correction lookup table 66) that associates the locations of the individual sensor units 25,28 with the alignment of the 2D ultrasound imaging plane. In order to determine the relationship between the sensor configuration reference frame and the coordinate system (reference frame) of the transducer imaging plane, several methods can be utilized. Existing methods are reviewed in L. Mercier, et al., “A review of calibration techniques for freehand 3-D ultrasound systems”, Ultrasound in Medicine and Biology, 31(2):143-165, 2005, and an automatic calibration method is described in R. W. Prager, et al., “Rapid calibration for 3-D freehand ultrasound”, Ultrasound in Medicine and Biology, 24(6):855-869, 1998. Both of these references are incorporated by reference in their entirety. The techniques described involve determining the relationship between imaged objects and the known spatial positions of the objects. In addition, the positioning and orientation errors can be measured by moving the transducer with the sensor configuration independently along each of the six degrees of freedom. If additional redundant degrees of freedom are available from extra sensors, then the processor uses the additional data for the evaluation of individual sensor alignment.

Registration processor 30 references the changes in position and orientation data relative to initial position and orientation coordinates 68 at a starting time. In other words, the starting coordinates are all zero and all subsequent tag data are relative to the position and orientation at starting time. In order to relate the sensor configuration coordinate system to changes in transducer movement and orientation, standard coordinate transformation methods (see B. Jahne, “Practical Handbook on Image Processing for Scientific and Technical Applications”, CRC Press, Boca Rotan, FLA, Chapter 8, 2004, incorporated by reference in relative part) in imaging processing are utilized. The changes in the sensor configuration coordinate system in terms of orientation and translation may be computed via a matrix multiplication (for angle changes) and/or addition (for position changes) of the previous location given the changes in the six degrees of freedom (translation parameters x, y, z, and rotation parameters α (rotation angle about the x axis), β (rotation angle about the y axis), and γ (rotation angle about the z axis). This computation is often performed as one combined matrix operation, referred to as a Jacobian.

Registration processor 30 preferably additionally has the capability to correct self-correct sensor drift and bias based on specific information 76 from the sensor manufacturer or through use of additional sensing elements. For example, in some embodiments, an auxiliary on-board temperature sensor 70 is continually polled by the registration processor 30 and, based on the manufacturer's sensor output characteristic with temperature (stored in an on-board table), the processor corrects the sensor output appropriately. Other auxiliary sensors may aid registration processor 30 in sensing changes, such as DC bias drift, and correct 3D tag data as needed.

The registration processor 30 receives timing data from clock 20, in order to coordinate the reception of the position and angle signals, compensation of the obtained position and orientation data, and geometric transformation and correction, as necessary into 3-D tag information that is supplied as a continuous stream 72 of 3D position data as a function of time to the imaging system. The various sensor outputs are sampled (and interpolated, if necessary) according to a clock signal, so that stream 72 of tag data is continuous and synchronized. Additionally, the timing of the position data acquisition is synchronized with the transmission of radio frequency pulse echo data 74 from the transducer 12. Alternatively, the registration processor 30 can function in a different mode in which it will send 3-D position tag information only when requested via a request signal 52 by the imaging system 24 at the start or completion of a 2D frame.

Calibration

Optionally, the relative positions of the sensors and the transducer image scan plane can be determined through use of known methods for calibrating free-hand 3D ultrasound equipment, such as described by R. W. Prager, R. N. Rohling, A. H. Gee, and L. Berman. Rapid calibration for 3-D freehand ultrasound. Ultrasound in Medicine and Biology, 24(6):855-869, 1998 and L. Mercier, T. Lango, F. Lindseth and L. D. Collins. A review of calibration techniques for freehand 3-D ultrasound systems. Ultrasound in Medicine and Biology, 31(2):143-165, 2005, the contents of which are hereby incorporated by reference. Spatial calibration, generally, involves scanning a known object from a variety of orientations—this can be a single point, a set of points, a cross-wire, a ‘z-shape’, a real or virtual plane, or in fact any known shape. By constraining the 3D reconstruction to match the known geometry of the scanned object, it is possible to derive a system of equations for spatial calibration parameters, or sensor data correction factors, that registration processor 30 can apply, as appropriate, to the received sensor data in order to improve accuracy. Embodiments of the invention may utilize such techniques to derive the geometric correction factors described above for the positions of said at least one said position sensing elements and/or said angle-sensing elements relative to the imaging plane and axes of a coordinate system associated with the degrees of freedom.

Sensor State Change Error Compensation

Optionally, as noted above, the registration processor may also compensate for sensing errors due to a change in the state of the sensing elements. For example, sensor errors may be due to drift and/or hysteresis. A temperature sensor providing input into registration processor 30 permits the processor to look up in the sensor correction lookup table geometric factors for application to the received sensor data. Temperature-dependent sensor characteristics are typically known a priori and supplied by sensor manufacturers. Another example is sensing and correcting for changes in the D.C. bias level.

Experiments

In an implementation of the invention was constructed by the applicants that utilized two WINDOWS XP™ software applications, TERASON and SONOCUBIC, which have been developed for free-hand ultrasound scanning without a registration system. Sonocubic is a 3D ultrasound rendering software application which collects scan planes and stores them for 3D visualization. The added registration system included an optical sensor with DSP-processor that was interfaced to a computer via a USB-interface. A DLL made it possible to interface Sonocubic to the driver to the optical sensor and to provide Sonocubic with the position tags necessary to position the scan planes correctly.

As noted above, an AGILENT DNS-2610 optical scanner commonly found in computer mice was utilized as the position sensor. A few optical configurations were evaluated, a first in which an LED illuminated the surface to be imaged through an optical fiber bundle in the transducer, a second approach in which the surface was illuminated by an LED mounted near the surface and with a lens in front of the optical fiber, and a third that did not use a fiber bundle, rather a small custom housing was constructed for mounting a single lens in front of the optical sensor. Tracking was achievable using each approach, although the third proved preferable for reduced blurring effects.

The Sonocubic software was modified to utilize the position tag information, and to alter its internal interpolation algorithm. The position data was extracted using a mouse filter driver from the ADNS-2610 sensor output. The change in sensor position is continuously updated inside the mouse and a driver stack, which was operated in polled mode in order to access the mouse filter driver and acquire the change in position each time Sonocubic requested it.

Five different scans were made of a phantom using the transducer and registration system, carried out along a non-linear scan path, with an offset of approximately 1 cm from center. The scan planes were collected by the modified Sonocubic software application. A modified interpolation algorithm calculated the data values for the voxels in a main grid. the sequence of scan planes in the maingrid was then saved to an AVI-file for image enhancement in MATLAB. Volume determinations were made correctly, with the highest deviation being 6% from the actual phantom volume. The mean was at 1% above actual and the standard deviation was 3.72%.

Although the invention has been described with respect to various embodiments, it should be realized this invention is also capable of a wide variety of further and other embodiments within the spirit of the invention.

It is claimed:

1. A free-hand, three-dimensional ultrasound imaging registration system, comprising:
a transducer probe including a probe housing and an ultrasound array transducer operatively disposed in said probe housing so as to supply ultrasound waves to a region of interest, to receive over time ultrasound waves reflecting from the region of interest as a plurality of transducer signals that can be converted into 2D image planes, each of said transducer signals having an associated image acquisition time;
at least one position sensor operatively integrated within or upon said probe housing, said at least one position sensor for acquiring, as a function of time position data of the probe in at least one translational degree of freedom relative to a reference position and a starting time and for convening said acquired position data into at least one position signal;
at least one angle sensor operatively integrated within or upon said probe housing, said at least one angle sensor for acquiring, as a function of time angular data of the probe in at least one rotational degree of freedom relative to a reference orientation and a starting time and for converting said acquired angular data into at least one angular signal;
a processing unit adapted to receive said associated image acquisition times, said at least one position signal, and said at least one angular signal, and to compute therefrom a position tag for each of said 2D image planes; and
means for communicating said transducer signals, said at least one position signal, and said at least one angular signal from said transducer probe to said processing unit.

2. The ultrasound imaging registration system of claim 1, wherein said at least one position sensor and said at least one angle sensor acquire said position data and said orientation data, respectively, independently from external references.

3. The ultrasound imaging registration system of claim 1, wherein:
said at least one position sensor acquires said position data in three translational degrees of freedom;
said at least one angle sensor acquires said angular data in three rotational degrees of freedom; and
each position tag comprises a 3D position tag.

4. The ultrasound imaging registration system of claim 3, wherein:
said at least one position sensor comprises a three-axis MEMS accelerometer;
said at least one angle sensor comprises a rotational three axis gyro.

5. The ultrasound imaging registration system of claim 1, wherein said processing unit is adapted to compute said position tag through the steps of:
obtaining said position data and said angular data, from said at least one position signal and at least one angular signal, respectively;
deriving position tag coordinates from geometric transformations of the position data and orientation data relative to a reference position and a reference orientation as a function of time;
associating each 2D image plane with position tag coordinates, by comparing the image acquisition time associated with each 2D image plane with timing data corresponding to said position tag coordinates.

6. The ultrasound imaging registration system of claim 1, wherein at least one of said position sensors and said angle sensors is reversibly integrated within or upon said probe housing.

7. The ultrasound imaging registration system of claim 1, wherein said at least one position sensors includes at least one single axis sensor.

8. The ultrasound imaging registration system of claim 1, wherein said at least one position sensors includes at least one multiple axes sensor.

9. The ultrasound imaging registration system of claim 1, wherein said at least one angle sensor includes at least one sensor sensing rotation about a singular axis.

10. The ultrasound imaging registration system of claim 1, wherein said at least one angle sensor includes at least one sensor sensing rotation about multiple axes.

11. The ultrasound imaging registration system of claim 1, wherein said at least one angle sensors consists of one or more sensors selected from the group consisting of capacitive MEMS devices, gyroscopes and accelerometers.

12. The ultrasound imaging registration system of claim 1, wherein said at least one position sensors consists of one or more sensors selected from the group consisting of optical sensors and capacitive MEMS devices.

13. The ultrasound imaging registration system of claim 1, wherein said at least one position sensor comprises an optical position sensor including:
at least one light source for illuminating the region of interest with sufficient intensity such that light reflects from the region of interest;
an optical imaging means including at least one lens disposed in or upon the probe so as to receive light reflected from the region of interest in the form of an optical image; and
a light-sensitive image capture device for converting the optical image output from the lens into said position signal.

14. The ultrasound imaging registration system of claim 13, wherein said optical imaging means further includes an optical fiber bundle optically coupled between said at least one lens and said light-sensitive image capture device.

15. The ultrasound imaging registration system of claim 1, wherein said communication means comprises a wireless transmission circuit.

16. The ultrasound imaging registration system of claim 1, further comprising a means for calibrating the relative positions of said at least one said position sensors and said angle sensors.

17. A free-hand, three-dimensional ultrasound imaging registration system, comprising:
a transducer probe including a probe housing and an ultrasound array transducer operatively disposed in said probe housing so as to supply ultrasound waves to a region of interest, to receive over time ultrasound waves reflecting from the region of interest as a plurality of transducer signals that can be converted into 2D image planes, each of said transducer signals having an associated image acquisition time;
at least one position sensor operatively integrated within or upon said probe housing, said at least one position sensor for acquiring as a function of time position data of the probe in three translational degrees of freedom relative to a reference position and a starting time and for converting said acquired position data into at least one position signal;
a processing unit adapted to receive said associated image acquisition times and said at least one position signal, and to compute therefrom a position tag for each of said 2D image planes; and
means for communicating said transducer signals and said at least one position signal from said transducer probe to said processing unit.

18. The ultrasound imaging registration system of claim 17, wherein said at least one position sensor acquires said position data independently from external references.

19. The ultrasound imaging registration system of claim 17, wherein said at least one position sensor comprises a three-axis MEMS accelerometer.

20. The ultrasound imaging registration system of claim 17, wherein said processing unit is adapted to compute said position tag through the steps of:
obtaining said position data from said at least one position signal;
deriving position tag coordinates from geometric transformations of the position data relative to a reference position and a reference orientation as a function of time;
associating each 2D image plane with position tag coordinates, by comparing the image acquisition time associated with each 2D image plane with timing data corresponding to said position tag coordinates.

21. The ultrasound imaging registration system of claim 17, wherein said at least one position sensors is reversibly integrated within or upon said probe housing.

22. The ultrasound imaging registration system of claim 17, wherein said at least one position sensors includes at least one singular axis sensor.

23. The ultrasound imaging registration system of claim 17, wherein said at least one position sensors includes at least one multiple axes sensor.

24. The ultrasound imaging registration system of claim 17, wherein said at least one angle sensors consists of one or more sensors selected from the group consisting of optical sensors and capacitive MEMS devices.

25. The ultrasound imaging registration system of claim 17, wherein said at least one position sensor comprises an optical position sensor including:
at least one light source for illuminating the region of interest with sufficient intensity such that light reflects from the region of interest;
an optical imaging means including at least one lens disposed in or upon the probe so as to receive light reflected from the region of interest in the form of an optical image; and
a light-sensitive image capture device for converting the optical image output from the lens into said position signal.

26. The ultrasound imaging registration system of claim 25, wherein said optical imaging means further includes an optical fiber bundle optically coupled between said at least one lens and said light-sensitive image capture device.

27. The ultrasound imaging registration system of claim 17, wherein said communication means comprises a wireless transmission circuit.

28. The ultrasound imaging registration system of claim 17, further comprising a means for calibrating the relative positions of said at least one said position sensors.

29. Method of registration for 3D ultrasound scanning, comprising the steps of:
providing a transducer probe including a housing within which is operatively disposed an ultrasound array transducer for supplying ultrasound waves to a region of interest, receiving over time ultrasound waves reflecting from the region of interest as a plurality of transducer signals that can be converted into 2D ultrasound image planes, each 2D ultrasound image plane having an associated image acquisition time, said transducer probe further including at least one position sensor and at least one angle sensor, the at least one position sensor and at least one angle sensor each operatively integrated within or upon said transducer housing and adapted to acquire as a function of time position data of the transducer probe in at least one translational degree of freedom relative to a reference position and a starting time, and angular data of the transducer probe in at least one rotational degree of freedom relative to a reference orientation and the starting time;
acquiring position data and orientation data of said transducer probe as a function of time relative to the reference position and reference orientation via the at least one position sensor and at least one angle sensor;
acquiring transducer signals in order to derive a sequence of 2D ultrasound image planes via the ultrasound array transducer; and
computing as a function of time from said acquired position data, said orientation data, and acquisition times associated with said sequence of 2D ultrasound image planes a position tag for the transducer probe.

30. The method of claim 29, wherein said at least one position sensor and said at least one angle sensor acquire said position data and said orientation data, respectively, independently from external references.

31. The method of claim 29, wherein said computing step further comprises the step of interrogating said at least one position sensor and said at least one angle sensor in a synchronous manner with the acquisition of said transducer signals.

32. The method of claim 29, further comprising the step of transmitting the computed position tags, ultrasound transducer signals and associated acquisition timing data to an ultrasound image display program.

33. The method of claim 29, wherein said computing step further comprises the steps of:
deriving tag position coordinates from geometric transformations of the position data and orientation data relative to the reference position and reference orientation as a function of time;
associating each 2D image plane with tag position coordinates, by comparing the image acquisition time for each 2D image plane with timing data corresponding to said tag position coordinates.

34. The method of claim 29, wherein acquiring the position data of the transducer probe comprises the steps of:
illuminating the region of interest with at least one light source with sufficient intensity such that light reflects from the region of interest;
receiving light reflected from the region of interest via an optical imaging means including at least one lens disposed in or upon the probe in the form of an optical image; and
converting the received optical image via a light-sensitive image capture device into said position signal.

35. The method of claim 29, further comprising the step of calibrating the relative positions of said at least one said position sensors and said angle sensors.

36. The method of claim 29, further comprising the step of compensating for sensing errors due to a change in state of said at least one position sensor or at least one angle sensor.

37. Method of registration for 3D ultrasound scanning, comprising the steps of:
providing a transducer probe including a housing within which is operatively disposed an ultrasound array transducer for supplying ultrasound waves to a region of interest, receiving over time ultrasound waves reflecting from the region of interest as a plurality of transducer signals that can be converted into 2D ultrasound image planes, each 2D ultrasound image plane having an associated image acquisition time, said transducer probe further including at least one position sensor operatively integrated within or upon said transducer housing and adapted to acquire as a function of time position data of the transducer probe in three translational degrees of freedom relative to a reference position and a starting time;
acquiring position data of said transducer probe as a function of time relative to the reference position via the at least one position sensor;
acquiring transducer signals in order to derive a sequence of 2D ultrasound image planes via the ultrasound array transducer; and
computing as a function of time from said acquired position data and acquisition times associated with said sequence of 2D ultrasound image planes a position tag for the transducer probe.

38. The method of claim 37, wherein said at least one position sensor acquires said position data independently from external references.

39. The method of claim 37, wherein said computing step further comprises the step of interrogating said at least one position sensor in a synchronous manner with the acquisition of said transducer signals.

40. The method of claim 37, further comprising the step of transmitting the computed position tags, ultrasound transducer signals and associated acquisition timing data to an ultrasound image display program.

41. The method of claim 37, wherein said computing step further comprises the steps of:
deriving tag position coordinates from geometric transformations of the position data relative to the reference position as a function of time;
associating each 2D image plane with tag position coordinates, by comparing the image acquisition time for each 2D image plane with timing data corresponding to said tag position coordinates.

42. The method of claim 37, wherein acquiring the position data of the transducer probe comprises the steps of:
illuminating the region of interest with at least one light source with sufficient intensity such that light reflects from the region of interest;
receiving light reflected from the region of interest via an optical imaging means including at least one lens disposed in or upon the probe in the form of an optical image; and
converting the received optical image via a light-sensitive image capture device into said position signal.

43. The method of claim 37, further comprising the step of calibrating the relative position of said at least one said position sensor.

44. The method of claim 37, further comprising the step of compensating for sensing errors due to a change in state of said at least one position sensor.
